(19) United States
(12) Patent Application Publication
(10) Pub. No.: US 2024/0423631 A1
(43) Pub. Date: Dec. 26, 2024

(54) BLOOD FLOW SUPPRESSION DEVICE AND METHOD

(71) Applicant: Walter Franz Poppenberger, Toronto (CA)

(72) Inventor: Walter Franz Poppenberger, Toronto (CA)

(21) Appl. No.: 18/749,885

(22) Filed: Jun. 21, 2024

(30) Foreign Application Priority Data
Jun. 21, 2023 (CA) 3202452

(51) Int. Cl. A61B 17/12; A61B 17/00

(52) U.S. Cl. CPC A61B 17/12136; A61B 17/1204; A61B 17/12099; A61B 2017/00548; A61B 2017/00557; A61B 2017/12004

(57) ABSTRACT

A battlefield injury suppression device preventing exsanguination. This intervention device is to treat specific injuries such as internal bullet or fragmentation wounds causing rapid blood loss. Imagine a stubby soda can only 3.5 cm tall with a pencil shaped rod of 12 cm sticking out of the bottom center, the upper portion is connected to a short access tube for a small gas cartridge. A soldier with minimal training will insert the tip to the full length of the device inside a bullet wound. Firmly holding it against the body and triggering a gas expansion process turning the pencil shaped rod into a balloon building up pressure against the injured tissue thus reducing or stopping hemorrhaging. The balloon expansion process is visible via an electronic digital pressure gauge and stopped manually at a given number. The procedure will extend the patient's life permitting a surgeon to intervene.

### CROSS-REFERENCE TO RELATED APPLICATION

This application claims priority to Canadian Patent Application Number 3,202,452, filed Jun. 21, 2023, the entirety of which is hereby incorporated by reference.

### FIELD

The description relate generally to blood flow control, particularly to blood flow suppression.

### BACKGROUND

Wounds can bleed profusely. For example, battlefield conditions create mortal wounds that bleed profusely, and wars, such as the war in Ukraine, result in many such wounds.

### SUMMARY

HemoStop is intended as an injury blood suppression device, such as a battlefield injury blood suppression device, to prevent exsanguination. This first assembly is the basic idea for various sizes, to treat specific injuries such as internal bullet or fragmentation injuries that cause rapid blood loss.

### BRIEF DESCRIPTION OF THE DRAWINGS

The drawings included herewith are for illustrating various examples of apparatus of the present specification and are not intended to limit the scope of what is taught in any way. In the drawings:

FIG. 1 is a side elevation view of a device to stop rapid blood loss;

FIG. 2 is a top view of the device of FIG. 1;

FIG. 3 is an expanded view of the top view of FIG. 2;

FIG. 4 is a side view of a top portion of the device of FIG. 1;

FIG. 5 is an enlarged view of individual parts including discs;

FIG. 6 is an exploded view of a bottom portion of the device of FIG. 1; and,

FIG. 7 is a combination view showing portions of the views of FIG. 5 and FIG. 6.

### DETAILED DESCRIPTION

HemoStop is a device to stop rapid blood loss. HemoStop is a device inserted into wounds, such as bullet or fragmentation wounds, and the device is inflatable, pressurized to stop bleeding. The purpose of the HemoStop device is a first aid blood suppression device to prevent exsanguination and extends time for medical intervention. The disclosure will also be beneficial for ambulance and police services internationally.

HemoStop, a simple unforgettable shape consisting of a 12 cm pencil shaped rod protruding from the bottom center of a 3.5 cm tall stubby soda can shaped plastic canister. The top of the canister holds a rectangular digital pressure gage and a gas cartridge attached to a gas feeder tube assembly leading to the internal part fittings to inflate the 12 cm pencil shaped rod which consequently transforms into a pressurized balloon.

The rolled-up pencil shaped inflatable balloon of a condom type material should be rugged and hard enough to penetrate the same path a bullet or fragmentation splinter created. Currently the device, if manufactured as per the drawing and specifications, needs to be tested on an animal possibly under strict humane circumstances without pain. One of my fears is an expansion failure when the pressurized balloon is pierced by sharp shrapnel that originally caused the injury inside the body. In that case, there will be pressure initially on the digital gage and possibly a little later no pressure, in that case, one must pull the previously inserted inflatable device out of the body by about 5 cm, stab the visible neck of the balloon with the tip of a scalpel or knife supplied with the HemoStop device, and cut half of the visible neck to facilitate the insertion of a second device directly into the failed tubing while pulling the opened neck sideways as the new device is inserted as far as possible. Now cut the remaining piece of the neck without damaging the new HemoStop device and repeat the inflation process.

Chances are after testing, a modified rubber compound will prevent a shrapnel puncture of the expandable balloon. Nevertheless, I have a modification to make the base of the housing detachable, held in place by a latch against a seal and two clamps, which would facilitate a quicker re-insertion of the second HemoStop device if shrapnel deflation would become a problem.

The HemoStop device is shown in drawings, one of which is complete and three pages are with individual sections of the complete drawing slightly enlarged. A first page shows the pencil shaped rod glued to the inside of the 10 mm center hole of the 60 mm diameter base of the gas pressure control housing. The loose rubber with the rolled-up lip which exits the opposite side of the glued pencil shaped rod will be attached to the 16 mm disc that will press against a matching depression in the 50 mm cage disc attachment plate glued in the Main housing and creating a perfect gas seal. A second page shows a top view of the housing showing the gas pressure indicator and the gas feeder assembly for the gas supply and pressure regulation, available commercially. The side view gives a detailed view of the housing and the glued in gas seal attachment point, currently the 16 mm disc sits loose just held by tension from the rubber sleeve/lip assembly but may have to be centered and glued to give a proper fit during assembly. A third page shows an explanation of individual parts inclusive enlarged view of dimensions of discs.

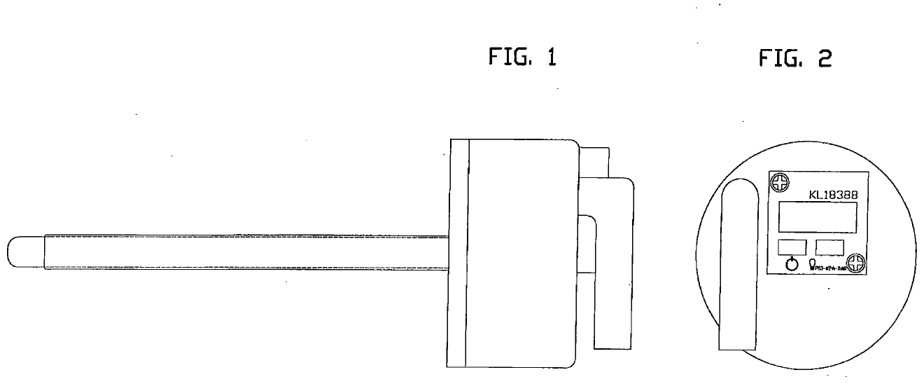

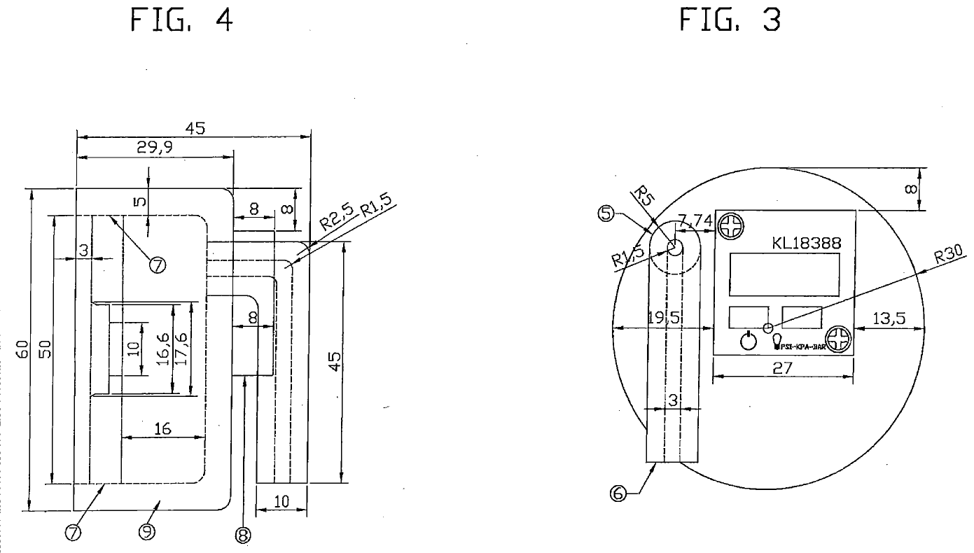

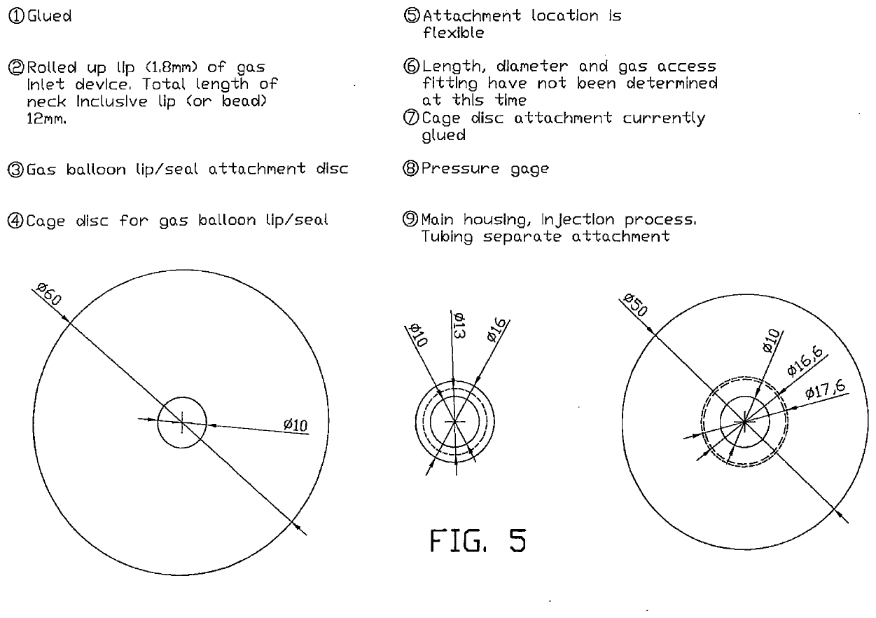

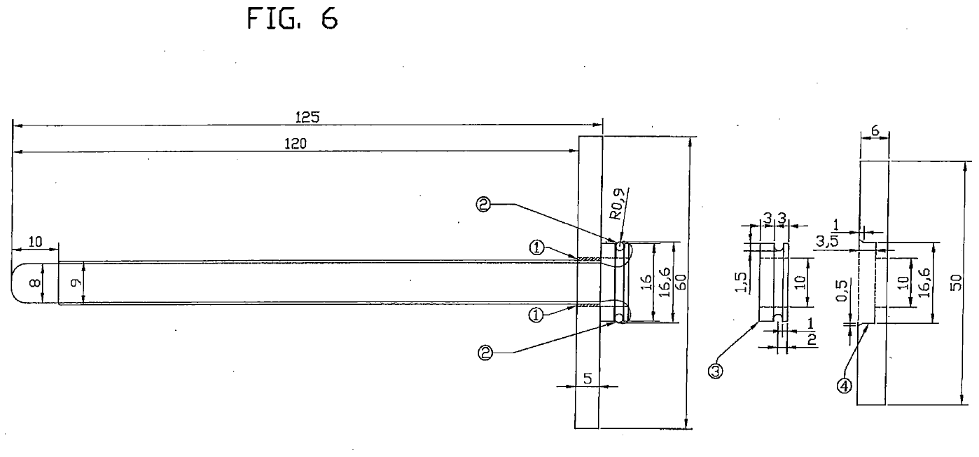

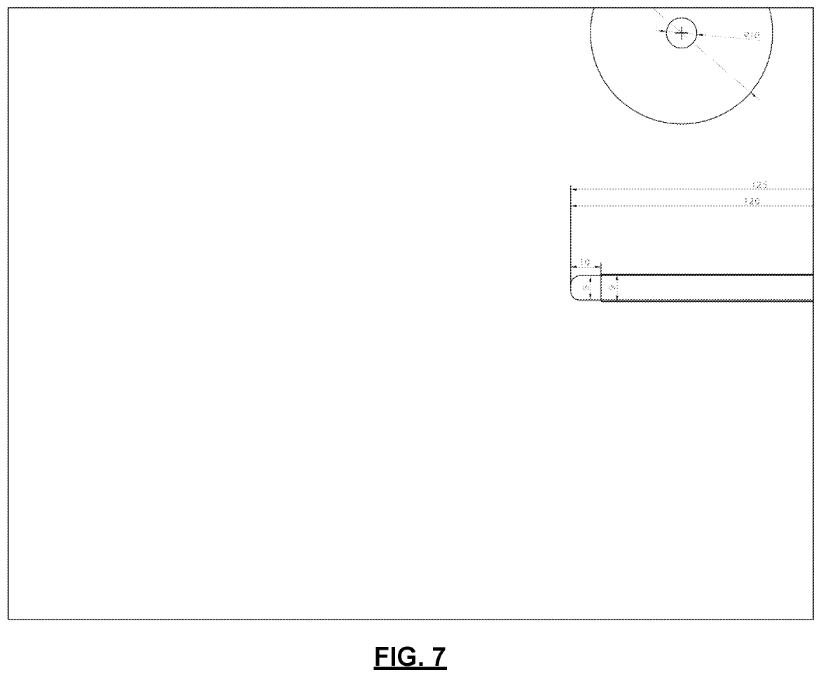

Referring now to FIGS. 1 to 7, illustrated is an example device. The front portion of the device A is a thin rod 120 mm long by 9 mm diameter centered inside a 60 mm disc B glued to a 60 mm diameter housing C, the total length of the housing inclusive a 15 mm gas inlet tubing is 45 mm. The front lifesaving segment of the device A is pencil shaped on one end and consists of a tightly longitudinally rolled-up expandable balloon D of a condom type material, all except the 8 mm tip D of the 12.5 cm long compressed balloon assembly is stored inside a stabilizing tube E like an open-ended sleeve for an umbrella. The distal loose neck F of the balloon is an additional 12 mm in length with a 12 mm gas entrance ending with a rolled up 1.8 mm diameter lip G. The assembly of the device starts by passing the 8 mm tip D through the center hole of the 16 mm plastic disc H starting on the side with the external groove I, one end of the loose lip is stretched and fitted over the external groove of the disc and will serve ultimately as a gas seal. Next, pass the tip of the device with the 16 mm disc attached through the center hole J of the 60 mm disc with a thickness of 5 mm serving as a base.

Pulling the inflatable device against the base disc will stretch the neck of the balloon far enough to apply a 2 mm circular bead of glue K at 5 mm to 3 mm away from the distal end of the stabilizing tube. Slowly release pressure against the base disk and slide the device to align the distal end of the stabilizing tube coated with glue towards the edge of the opposite internal end of the 60 mm base. Manufacturing the upper housing in large quantities utilizes a standard plastic injection process.

Initially a stabilizing centering cage L glued inside the main housing will loosely fit over the 16 mm lip/disc H and will create a gas tight seal when compressed by the external cover during final assembly. Pressing and gluing the external cover assembly to the base plate will create the desired bond initially; however, with a modification in mind the same outcome can be achieved by tightly screwing both ends against a Teflon seal. I suggest coating the total area of the inflatable balloon with a styptic drug externally as it will induce vasoconstriction when the inflating balloon creates counter pressure against damaged veins and arteries in the injured cavity. In addition, a safe small amount of non-toxic lubricant inside the balloon neck may help the inflation process.

The external tubular attachment device for the gas supply has not been determined at this time since there are various commercial attachments to inflate the balloon. The triggered inflation device should have just enough pressure to rupture the balloon stabilization sleeve designed to tear along the pre-treated punctured longitudinal axis and permit the balloon to inflate inside and against the injured cavity through a one-way air valve. The proximal stabilizing sleeve of the inflation device glued to the 10 mm hole of the base disc during final assembly was for one purpose only, permitting all parts of the device to be removable inclusive the ripped stabilizing tube when the surgeon releases the one-way pressure valve or on the sphygmomanometer.

If fine tuning internal balloon pressure with the pressure regulator of the gas cartridge is not reliable, adding one additional attachment provision at the gas supply line for a sphygmomanometer to adjust the counter pressure against vascular and tissue bleeding in the wound cavity. I assume even a small steady sign of blood pressure read from a pulse oximeter attached to a cell phone would indicate the balloon is arresting blood loss.

The inflation device should insert gas through the 90° elbow thus allowing better access by the field rescue operator. There are various options to achieve rapid inflation of the HemoStop device but I do not know if hot gases from a miniature airbag explosive cartridge will be acceptable or safe for the body and balloon. Another option is a CO2 cartridge serving as an inflation device but I do not know if freezing gas will negatively affect the injury, however it also could be temporarily helpful by nearly flash freezing the tissue. Another option is Helium or Nitrous gas cartridges from AUTOLIVE.

The above device may find its way into every emergency response kit universally. A comrade in arms with minimal training can likely accomplish inserting the HemoStop tip all the way inside a bullet wound, the device should be pressed against the body while triggering gas flow. Carefully watching the red image of an electronic digital pressure gage and stopping gas expansion at a given pressure.

Various embodiments have been described herein by way of example only. Various modification and variations may be made to these example embodiments without departing from the invention, which is limited only by the appended claims.

1. A device for minimizing blood loss from a penetrating injury to a body comprising:
a housing comprising a balloon securing portion;
a gas channel running through the housing and the balloon securing portion, between a gas inlet on a first face of the housing and a gas outlet on a second face of the housing;
a stabilizing tube in communication with the gas channel, extending from the balloon securing portion and gas outlet.

2. The device of claim 1, wherein the balloon securing portion comprises:
a hollow disc, having a circumferential external groove configured to receive a lip of a balloon; and
a recess within the housing configured to receive the disc and the lip of the balloon.

3. The device of claim 2, wherein the recess comprises a channel in communication with each of the stabilizing tube and the gas channel.

4. The device of claim 2, wherein the housing comprises two separable portions:
an upper portion comprising the recess; and
a lower portion.

5. The device of claim 1, further comprising a pressure gauge in connection with the gas channel.

6. The device of claim 1, further comprising gas inlet tubing in connection with the gas inlet.

7. The device of claim 1, wherein the stabilizing tube is perforated along its length.

8. The device of claim 1, wherein the stabilizing tube is 11 cm long.

9. The device of claim 1, wherein the stabilizing tube is 9 mm in diameter.

10. A kit for minimizing blood loss from a penetrating injury to a body comprising:
the device of claim 1; and
a balloon.

11. The kit of claim 10, further comprising a triggered inflation device.

12. A method of minimizing blood loss from a penetrating injury to a body using the device of claim 1 comprising:
feeding a balloon through the stabilizing tube;
securing a lip of the balloon within the device at the balloon securing portion;
inserting the stabilizing tube into the body through a hole created by the penetrating injury;
connecting a triggered inflation device to the gas channel; and
inflating the balloon by activating the triggered inflation device.

13. The method of claim 12, wherein the balloon securing portion comprises:
a hollow disc, having a circumferential external groove configured to receive the lip of the balloon; and
a recess within the housing configured to receive the disc and lip of the balloon.

14. The method of claim 13, wherein the step of securing the balloon within the device at the balloon securing portion comprises folding the lip of the balloon over a top end of the hollow disc and lodging the lip of the balloon in the external groove.

15. The method of claim 12, further comprising coating the balloon with a styptic drug.
